(19) United States
(12) Patent Application Publication
(10) Pub. No.: US 2017/0119775 A1
(43) Pub. Date: May 4, 2017

(54) TREATMENT OF COGNITIVE DISORDERS

(71) Applicant: Algiax Pharmaceuticals GmbH, Erkrath (DE)

(72) Inventors: Guido Koopmans, Erkrath (DE); Birgit Hasse, Erkrath (DE)

(21) Appl. No.: 15/129,448

(22) PCT Filed: Mar. 20, 2015
(86) PCT No.: PCT/EP2015/055989

§ 371 Date: Sep. 27, 2016

Related U.S. Application Data
(60) Provisional application No. 61/971,799, filed on Mar. 28, 2014.

(30) Foreign Application Priority Data
Mar. 28, 2014 (EP) 14001150.3

(51) Int. Cl. A61K 31/53

(52) U.S. Cl. CPC A61K 31/53

(57) ABSTRACT

The technology provided herein relates to the novel use of compounds for improving cognition, concentration capacity, learning capacity and/or memory retentiveness, in particularly for the treatment and/or prophylaxis of cognitive, concentration capacity, learning capacity and/or memory retentiveness disorders.

### FIELD OF THE DISCLOSURE

The technology provided herein relates to the novel use of compounds like 7-(4-tert-butylcyclohexyl)-imidazotriazinones for improving cognition, concentration capacity, learning capacity and/or memory retentiveness, in particularly for the treatment and/or prophylaxis of cognitive, concentration capacity, learning capacity and/or memory retentiveness disorders.

### BACKGROUND

Cognitive failure (dysfunction or loss of cognitive functions, the process by which knowledge is acquired, retained and used) commonly occurs in association with central nervous system (CNS) disorders or conditions, including age-associated memory impairment, delirium (sometimes called acute confusional state), dementia (sometimes classified as Alzheimer's or non-Alzheimer's type), Alzheimer's disease, Parkinson's disease, Huntington's disease (chorea), mental retardation (e.g. Rubenstein-Taybi Syndrome), cerebrovaslular disease (e.g. stroke, ischemia), affective disorders (e.g. depression), psychotic disorders (e.g., schizophrenia, autism (Kanner's Syndrome)), neurotic disorders (i.e. anxiety, obsessive-compulsive disorder), attention deficit disorder (ADD), subdural hematoma, normal-pressure hydrocephalus, brain tumor, head or brain trauma.

Cognitive dysfunction causes significant impairment of social and/or occupational functioning, which can interfere with the ability of an individual to perform activities of daily living and greatly impact the autonomy and quality of life of the individual.

Diminished cognitive processes refer to the difficulties with attention, learning, memory and executive function (relevant reactions to external stimuli). These can include: deficits in attention, disorganized thinking, slow thinking, difficulty in understanding, poor concentration, impairment of problem solving, poor memory, difficulty in expressing thoughts and/or difficulty in integrating thoughts, feelings and behaviour and extinction of irrelevant thoughts as well as attention and vigilance, verbal learning and memory, visual learning and memory, speed of processing and social cognition.

Phosphodiesterases (E.C. 3.1.4.17) are a class of enzymes that catalyze the hydrolysis of the 3′-phosphodiester bond of 3′, 5′-cyclic nucleotides. The phosphodiesterase 4 (PDE4) isoform specifically hydrolyzes adenonsine 3′, 5′ cyclic monophosphate (cAMP) to form 5′-adenosine monophosphate (5′-AMP). cAMP is a well-studied intracellular second messenger that is known to be responsible for regulating a number of cellular processes including transcriptional regulation. One signaling pathway known to be regulated by intracellular levels of cAMP is the CREB pathway. The CREB pathway is responsible for regulating transcriptional activity in the brain (including the hippocampus) that leads to protein syntheses required for learning and memory, especially the consolidation of short-term to long-term memory. It is known that inhibition of PDE4 improves cognitive function in mammals, including contextual memory and object recognition (Tully, et. al., Nature Reviews Drug Discovery, 2003, 2, 267-277; and Barad, et al., Proc. Natl. Acad. Sci. 1998, 95, 15020-15025). It has also been shown to improve memory in animals with impaired CREB function (see Bourtchouladze, et. al., Proc Natl Acad Sci USA, 2003, 100, 10518-10522).

Numerous companies have invested in the development of specific PDE4 inhibitors to treat a variety of diseases, most notably in the anti-inflammatory field (e.g. Rolipram™, and Ariflo™) Challenges that are facing the PDE4 inhibitors are mainly nausea, vomiting, increased gastric acid secretion which may be because of selectivity towards binding sites. Based on the prior art reports, compounds with selectivity for the high-affinity rolipram binding site causes side effects whereas compounds with selectivity for low-affinity rolipram binding site are expected to have better therapeutic effects compared to rolipram (J Biol. Chem. 1992, 267(3): 1798-1804; J. Biol. Chem. 1999, 274(17):11796-11810).

The cognitive and functional decline observed in Alzheimer's patients has also been attributed to a cholinergic deficiency in the central nervous system. At least four drugs that have been used to treat Alzheimers Disease, i.e. tacrine, donepezil (donepeZil HCL; 1-benyZl-4-[(5,6-dimethoxy-1-indanon)-2-yl]methylpiperidine monohydrochloride), rivastigmine ((S)iN-Ethyl-Nmethyl-3-[1-(dimethylamino)ethyl]-phenyl carbamate) and galantamine (galantamine hydrobromide; (4aS,6R,8aS)-4a, 5,9,10,1 1,12-hexahydro-3-methoxy-1 1-methyl-6H-benZo furo[3a,3,2-ef][2]benZaZepin-6-ol hydrobromide), appear to act as acetylcholinesterase inhibitors that increase acetylcholine in the CNS.

Thus, there is currently a need for compounds that are useful for improving cognitive function in humans but cause little or no side effects, in particular that cause little or no emesis.

### SUMMARY OF THE DISCLOSURE

The present disclosure pertains to specific PDE4-inhibitors with a binding profile showing a higher affinity to the low-affinity rolipram binding site than to the high-affinity rolipram binding site (ratio>100 and higher) which are expected to induce gastrointestinal toxicity less prone as other PDE4-inhibitors like rolipram and by that widen the potential therapeutic window.

In a first aspect, embodiments of this disclosure provide compounds for improving cognition, concentration capacity, learning capacity and/or memory retentiveness.

The present disclosure relates to compounds that inhibit PDE4 and that are useful to improve cognitive function. Accordingly, in one embodiment a compound of the present disclosure is a compound of the formula I:

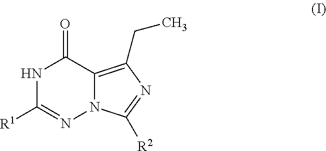

wherein:
i) R¹ denotes (C₆-C₁₀)-aryl, which is optionally substituted by identical or different residues selected from the group consisting of halogen, (C₁-C₄)-alkyl, trifluoromethyl, cyano, nitro and trifluoromethoxy, or denotes (C₁-C₈)-alkyl, which is optionally substituted by 3- to 10-membered carbocyclyl, or denotes 3- to 10-membered carbocyclyl, which is optionally substituted by identical or different (C₁-C₄)-alkyl residues, and R² denotes 4-tert-butyl-cyclohex-1-yl, or
ii) R¹ denotes naphthyl, or denotes phenyl, which is optionally substituted by identical or different halogen atoms, and R² denotes 4-tert-butyl-cyclohex-1-yl, or
iii) R¹ denotes (C₆-C₁₀)-aryl, which is optionally substituted by identical or different residues selected from the group consisting of halogen, (C₁-C₄)-alkyl, trifluoromethyl, cyano, nitro and trifluoromethoxy, or denotes (C₁-C₈)-alkyl, which is optionally substituted by 3- to 10-membered carbocyclyl, or denotes 3- to 10-membered carbocyclyl, which is optionally substituted by identical or different (C₁-C₄)-alkyl residues, and R² denotes cis-4-tert-butylcyclohex-1-yl, or
iv) R¹ denotes naphthyl, or denotes phenyl, which is optionally substituted by identical or different halogen atoms, and R² denotes cis-4-tert-butylcyclohex-1-yl, for the use in treating diminished cognitive processes in cognitive, concentration capacity, learning capacity and/or memory retentiveness disorders.

In a further aspect, the present disclosure relates to compounds for the use in treating diminished cognitive processes in cognitive, concentration capacity, learning capacity and/or memory retentiveness disorders, wherein the compound is a 7-(4-tert-butylcyclohexyl)-imidazotriazinone.

In particular, the compound is a compound of the formula (II)

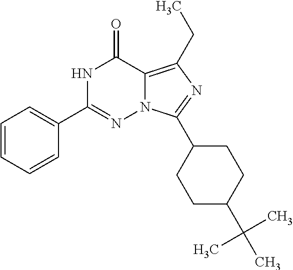 [SMILES: CCc1nc(C2CCC(C(C)(C)C)CC2)n2nc(-c3ccccc3)[nH]c(=O)c12]

or a pharmaceutically acceptable salt, solvate or stereoisomer thereof.

The disclosure also provides a pharmaceutical composition comprising a compound of formula I and/or formula II, or a pharmaceutically acceptable salts thereof, in combination with a pharmaceutically acceptable diluent or carrier.

The disclosure also provides a therapeutic method for improving cognitive function in an animal comprising administering to the animal an effective amount of a compound of formula I, or a pharmaceutically acceptable salt thereof.

The disclosure also provides a method for inhibiting PDE4 receptors (in vitro or in vivo) comprising contacting the receptors with an effective inhibitory amount of a compound of formula I and/or formula II, or a pharmaceutically acceptable salts thereof.

The present disclosure provides a compound of formula I or formula II, or a pharmaceutically acceptable salt thereof for use in medical therapy (e.g. for use in improving cognitive function or for use in treating a disease or condition wherein inhibition of PDE4 receptor function is indicated or for treating a psychiatric disorder), as well as the use of a compound of formula I or formula II for the manufacture of a medicament useful for improving cognitive function in an animal, in particular in human.

The disclosure also provides a pharmaceutical composition comprising a compound of formula I and/or formula II, or a pharmaceutically acceptable salt thereof in combination with an acetylcholinesterase inhibitor (e.g., donepezil or rivastigmine). The method can improve cognition in patients that have already benefited from an increase in one or more aspects of cognition stemming from the administration of an acetylcholinesterase inhibitor. Thus, a patient already benefiting from acetylcholinesterase inhibitor in one or more aspect of cognition can gain further benefit in one or more aspects of cognition from administration of 7-(4-tert-butylcyclohexyl)-imidazotriazinone and a pharmaceutically acceptable salts thereof.

The disclosure also provides a pharmaceutical composition comprising a compound of formula I and/or formula II, or a pharmaceutically acceptable salt thereof in combination with an acetylcholinesterase inhibitor (e.g., donepezil or rivastigmine) both administered at a subclinical dose (i.e., a dose that does not improve memory). Thus, a patient can experience a benefit (e.g., improved memory or cognition) from a combination of drugs each of which is administered at very low, side-effect reducing or side-effect avoiding dose. Moreover, the combination of drugs may provide a benefit for a wider range of patients and/or over a longer period of treatment. In the case of administering a dose that is subclinical, 7-(4-tert-butylcyclohexyl)-5-ethyl-2-phenylimidazo[5,1-f][1,2,4]triazin-4(3H)-one or a pharmaceutically acceptable salt thereof can be used at a daily oral dose of less than 0.3 mg/kg, 0.1 mg/kg, 0.05 mg/kg, 0.03 mg/kg or 0.01 mg/kg.

For donepizil, the daily dose used with 7-(4-tert-butylcyclohexyl)-5-ethyl-2-phenylimidazo[5,1-f][1,2,4]triazin-4(3H)-one or a pharmaceutically acceptable salt thereof can be 10 mg, 5 mg, 4.5 mg, 4 mg, 3.5 mg, 3 mg, 2.5 mg, 2 mg, 1 mg or 0.5 mg. The daily dose can be between 5 and 0.5 mg (e.g., 4.5-1.0 mg/day, 4.5-2.0 mg/day, 4.0-2.0 or 2.5 mg/day). For rivistigmine the daily dose for use in combination can be 11, 10, 9, 8, 7, 6 or 5 mg. For galantamine the daily dose for use in combination can be 20, 15, 13, 12, 11, 10, 9, 8, 7, 6 or 5 mg.

In still another aspect, embodiments of this disclosure provide compounds for the preparation of a medicament for improving cognition, in particularly for the treatment and/or prophylaxis of cognitive, concentration capacity, learning capacity and/or memory retentiveness disorders

In a further aspect, embodiments of this disclosure relate to methods of treating cognitive impairment, which comprise administering to a patient in need of such treatment a therapeutically effective amount of a compound according to this disclosure.

Further, embodiments of this disclosure relates to imidazotriazinones derivatives like 7-(4-tert-butylcyclohexyl)-imidazotriazinones, pharmaceutically acceptable salts, solvates, hydrates, stereoisomers, clathrates, or prodrugs thereof for use in the treatment of cognitive, concentration capacity, learning capacity and/or memory retentiveness disorders.

### BRIEF DESCRIPTION OF THE DRAWINGS

FIG. 1 is a diagram showing the effect of AP61 (p.o., immediately after T1) in a test of natural forgetting in the ORT. The dotted line indicates the SEM of the fictive group (mean: 0, SEM: 0.07). A difference from the fictive group showing no discrimination is depicted with an asterisk (t-test: * p<0.05).

FIG. 2 is a diagram showing the effect of 0.1 or 0.3 mg/kg rolipram in the xylazine/ketamine-induced anesthesia test (mean±SEM). Fifteen minutes after induction of anesthesia, rats received vehicle or rolipram (p.o., 2 ml/kg). Duration of anesthesia, expressed as a percentage, was assessed by the return of the righting reflex. A difference from vehicle is depicted with asterisks (post-hoc Bonferroni t-tests: * p<0.05, *** p<0.001).

FIG. 3 is a diagram showing the effect of administration of different doses of AP61 in the xylazine/ketamine-induced anesthesia test (mean±SEM). 3.5 h before induction of anesthesia, rats were treated with 0.03, 0.1, 0.3, 1.0 or 3.0 mg/kg AP61 (p.o., 2 ml/kg). Duration of anesthesia, expressed as a percentage, was assessed by the return of the righting reflex. A difference from vehicle is depicted with asterisks (post-hoc Bonferroni t-tests: * p<0.05, *** p<0.001).

FIG. 4 is a diagram showing the effect of co-administration of sub-efficacious doses of AP61 (0.01 mg/kg, p.o., 4 min after T1) and donepezil (0.1 mg/kg, p.o., 30 min before T1) in a test of natural forgetting in the ORT. The dotted line indicates the SEM of the fictive group (mean: 0, SEM: 0.07). A difference from the fictive group showing no discrimination is depicted with hashes (t-test: ### P=0.000). When compared with the vehicle+vehicle condition, the AP61+donepezil condition showed improved memory performance, as indicated by the repeated-measures ANOVA (***: P=0.001).

### DETAILED DESCRIPTION OF THIS DISCLOSURE

Disclosed herein is the use of 7-(4-tert-butylcyclohexyl)-imidazotriazinones, active metabolites and/or derivatives thereof for the treatment of diminished cognitive processes in cognitive, concentration capacity, learning capacity and/or memory retentiveness disorders.

Diminished cognitive processes can be experienced in several patient groups, e.g. by schizophrenic, depressive or psychotic patients and patients with attention deficit hyperactivity disorder (ADHD), Parkinson's disease, mild cognitive impairment (MCI), dementia, anxiety, age associated memory impairment, Alzheimer's Disease or post-traumatic stress disorder and in a range of neurodegenerative diseases in addition to Parkinson's Disease and Alzheimer's Disease.

Diminished cognitive processes refer to the difficulties with attention, learning, memory and executive function (relevant reactions to external stimuli). These can include: deficits in attention, disorganized thinking, slow thinking, difficulty in understanding, poor concentration, impairment of problem solving, poor memory, difficulty in expressing thoughts and/or difficulty in integrating thoughts, feelings and behaviour and extinction of irrelevant thoughts as well as attention and vigilance, verbal learning and memory, visual learning and memory, speed of processing and social cognition.

In an advantageous embodiment the specific compounds of the disclosure are imidazotriazinone derivatives and metabolites described in U.S. Pat. No. 6,610,687 B1, which is incorporated herein by reference.

Embodiments of the compounds according to the present disclosure are Imidazotriazinones of the general formula (I)

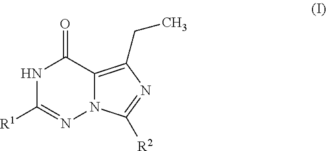

in which R¹ denotes (C₆-C₁₀)-aryl, which is optionally substituted by identical or different residues selected from the group consisting of halogen, (C₁-C₄)-alkyl, trifluoromethyl, cyano, nitro and trifluoromethoxy, or denotes (C₁-C₈)-alkyl, which is optionally substituted by 3- to 10-membered carbocyclyl, or denotes 3- to 10-membered carbocyclyl, which is optionally substituted by identical or different (C₁-C₄)-alkyl residues, and
R² denotes 4-tert-butyl-cyclohex-1-yl.

Another embodiment of the disclosure relates to the use according to the present disclosure of compounds of the general formula (I), in which

R¹ denotes naphthyl, or denotes phenyl, which is optionally substituted by identical or different halogen atoms and
R² has the meaning indicated above.

Another embodiment of the disclosure relates to the use of compounds of the general formula (I), in which R¹ has the meaning indicated above, and R² denotes cis-4-tert-butylcyclohex-1-yl.

The compounds according to this disclosure can also be present in the form of their salts, hydrates and/or solvates.

In advantageous embodiments, the compound used for the treatment of diminished cognitive processes in cognitive, concentration capacity, learning capacity and/or memory retentiveness disorders is a 7-(4-tert butyl-cyclohexyl)-imidazotriaziones.

A specific example of compounds used for the treatment of diminished cognitive processes in cognitive, concentration capacity, learning capacity and/or memory retentiveness disorders, but not limited to compounds with the following structure (formula II):

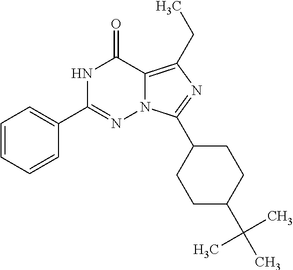 [SMILES: CCc1nc(C2CCC(C(C)(C)C)CC2)n2nc(-c3ccccc3)[nH]c(=O)c12]

In further advantageous embodiments, the compound is 7-(4-tert-butylcyclohexyl)-5-ethyl-2-phenylimidazo[5,1-f][1,2,4]triazin-4(3H)-one or a pharmaceutically acceptable salt, solvate or stereoisomer thereof.

In further advantageous embodiments, a compound according to the present disclosure is used as the only physically active compound in the treatment of diminished cognitive processes in cognitive, concentration capacity, learning capacity and/or memory retentiveness disorders without a second active agent.

In yet other advantageous embodiments, the disclosure relates to pharmaceutical compositions for the prophylaxis and/or treatment of diminished cognitive processes in cognitive, concentration capacity, learning capacity and/or memory retentiveness disorders, which comprises a therapeutically effective amount of a compound according to the present disclosure in admixture with a pharmaceutical acceptable carrier or excipient.

In advantageous embodiments, the pharmaceutical composition is used for the prophylaxis and/or treatment of diminished cognitive processes in cognitive, concentration capacity, learning capacity and/or memory retentiveness disorders, whereby the composition comprises a therapeutically effective amount of 7-(4-tert-butylcyclohexyl)-imidazotriazinones or a physiologically functional derivative thereof in admixture with a pharmaceutical acceptable carrier or excipient. In advantageous embodiments the pharmaceutical composition comprises 7-(4-tert-butylcyclohexyl)-5-ethyl-2-phenylimidazo[5,1-f][1,2,4]triazin-4(3H)-one or a pharmaceutically acceptable salt, solvate or stereoisomer thereof.

Compounds according to the disclosure can either be commercially purchased or prepared according to the methods described in the publications, patents or patent publications disclosed herein. Further, optically pure compositions can be asymmetrically synthesized or resolved using known resolving agents or chiral columns as well as other standard synthetic organic chemistry techniques. Compounds used in the disclosure may include compounds that are racemic, stereomerically enriched or stereomerically pure, and pharmaceutically acceptable salts, solvates, stereoisomers, and prodrugs thereof.

As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like. Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

Examples for physiologically acceptable salts can also be salts of the compounds according to this disclosure with inorganic or organic acids. Preferred salts are those with inorganic acids such as, for example, hydrochloric acid, hydrobromic acid, phosphoric acid or sulphuric acid, or salts with organic carboxylic or sulphonic acids such as, for example, acetic acid, maleic acid, fumaric acid, malic acid, citric acid, tartaric acid, ethanesulphonic acid, benzenesulphonic acid, toluenesulphonic acid or naphthalenedisulphonic acid. Preferred pyridinium salts are salts in combination with halogen.

As used herein, and unless otherwise specified, the term “solvate” means a compound of the present disclosure or a salt thereof that further includes a stoichiometric or non-stoichiometric amount of solvent bound by non-covalent intermolecular forces. Where the solvent is water, the solvate is a hydrate.

As used herein and unless otherwise indicated, the term “prodrug” means a derivative of a compound that can hydrolyze, oxidize, or otherwise react under biological conditions (in vitro or in vivo) to provide the compound. Examples of prodrugs include, but are not limited to, derivatives of compounds according to the present disclosure that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of immunomodulatory compounds of the disclosure that comprise —NO, —NO2, —ONO, or —ONO2 moieties. Prodrugs can typically be prepared using well-known methods, such as those described in Burger's Medicinal Chemistry and Drug Discovery, 172-178, 949-982 (Manfred E. Wolff ed., 5th ed. 1995), and Design of Prodrugs (H. Bundgaard ed., Elselvier, New York 1985). As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxymethyl, pivaloyloxymethyl, and pivaloyloxyethyl esters), lactonyl esters (such as phthalidyl and thiophthalidyl esters), lower alkoxyacyloxyalkyl esters (such as methoxycarbonyl-oxymethyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, [alpha]-amino acid amides, alkoxyacyl amides, and alkylaminoalkylcarbonyl amides. Examples of biohydrolyzable carbamates include, but are not limited to, lower alkylamines, substituted ethylenediamines, amino acids, hydroxyalkylamines, heterocyclic and heteroaromatic amines, and polyether amines.

As used herein, and unless otherwise specified, the term “stereoisomer” encompasses all enantiomerically/stereomerically pure and enantiomerically/stereomerically enriched compounds of this disclosure. Furthermore, the term “stereoisomer” includes also tautomers which are isomers of organic compounds that readily interconvert by a chemical reaction (tautomerization).

As used herein, and unless otherwise indicated, the term “stereomerically pure” or “enantiomerically pure” means that a compound comprises one stereoisomer and is substantially free of its counter stereoisomer or enantiomer. For example, a compound is stereomerically or enantiomerically pure when the compound contains 80%, 90%, or 95% or more of one stereoisomer and 20%, 10%, or 5% or less of the counter stereoisomer, in certain cases, a compound of the disclosure is considered optically active or stereomerically/enantiomerically pure {i.e., substantially the R-form or substantially the S-form) with respect to a chiral center when the compound is about 80% ee (enantiomeric excess) or greater, preferably, equal to or greater than 90% ee with respect to a particular chiral center, and more preferably 95% ee with respect to a particular chiral center.

As used herein, and unless otherwise indicated, the term “stereomerically enriched” or “enantiomerically enriched” encompasses racemic mixtures as well as other mixtures of stereoisomers of compounds of this disclosure {e.g., R/S=30/70, 35/65, 40/60, 45/55, 55/45, 60/40, 65/35 and 70/30). Various inhibitor compounds of the present disclosure contain one or more chiral centers, and can exist as racemic mixtures of enantiomers or mixtures of diastereomers. This disclosure encompasses the use of stereomerically pure forms of such compounds, as well as the use of mixtures of those forms. For example, mixtures comprising equal or unequal amounts of the enantiomers of a particular inhibitor compound of the disclosure may be used in methods and compositions of the disclosure. These isomers may be asymmetrically synthesized or resolved using standard techniques such as chiral columns or chiral resolving agents. See, e.g., Jacques, J., et ah, Enantiomers, Racemates and Resolutions (Wiley-Interscience, New York, 1981); Wilen, S. H., et al, Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E.L. Eliel, Ed., Univ. of Notre Dame Press, Notre Dame, Ind., 1972).

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

The term “physiologically functional derivative” as used herein refers to compounds which are not pharmaceutically active themselves but which are transformed into their pharmaceutical active form in vivo, i. e. in the subject to which the compound is administered. Examples of physiologically functional derivatives are prodrugs such as those described below in the present application.

The term “derivative” as used herein refers to a compound that is derived from a similar compound or a compound that can be imagined to arise from another compound, if one atom is replaced with another atom or group of atoms. The term “derivative” as used herein refers also to a compound that at least theoretically can be formed from the precursor compound (see Oxford Dictionary of Biochemistry and Molecular Biology. Oxford University Press. ISBN 0-19-850673-2.)

The disclosure is also directed to the use of compounds of the formula I or II and of their pharmacologically tolerable salts or physiologically functional derivatives for the production of a medicament for the prevention and treatment of diminished cognitive processes.

Methods and uses according to the present disclosure encompass methods of preventing, treating and/or managing diminished cognitive processes in cognitive disorders and related syndromes, but are not limited to, schizophrenic, depressive or psychotic patients and patients with attention deficit hyperactivity disorder (ADHD), Parkinson's disease, mild cognitive impairment (MCI), dementia, anxiety, age associated memory impairment, Alzheimer's Disease or post-traumatic stress disorder and in a range of neurodegenerative diseases in addition to Parkinson's Disease and Alzheimer's Disease.

The symptoms, conditions and/or symptoms associated with cognitive disorders include, but are not limited to attention, learning, memory and executive function (relevant reactions to external stimuli). These can include: deficits in attention, disorganized thinking, slow thinking, difficulty in understanding, poor concentration, impairment of problem solving, poor memory, difficulty in expressing thoughts and/or difficulty in integrating thoughts, feelings and behaviour and extinction of irrelevant thoughts as well as attention and vigilance, verbal learning and memory, visual learning and memory, speed of processing and social cognition.

The suitability of a particular route of administration of an compound according to the present disclosure employed for a particular active agent will depend on the active agent itself (e.g., whether it can be administered orally without decomposing prior to entering the blood stream) and the disease being treated. An advantageous embodiment of the route of administration for a compound according to the present disclosure is orally. Further routes of administration are known to those of ordinary skill in the art.

The dosage of therapeutically effective amount of at least one compound varies from and also depends upon the age and condition of each individual patient to be treated. In an embodiment of the present disclosure, the recommended daily dose range of a compound according to the present disclosure for the conditions and disorders described herein lies within the range of from about, a daily dose of about 0.5 mg-500 mg/body, preferable 1.5 mg-150 mg/body and more preferable 5.0 mg-50 mg/body of the active ingredient is generally given for preventing and/or treating this disease, and an average single dose of about 0.5 mg, 1.5 mg, 5.0 mg, 15 mg, 50 mg, 150 mg, 500 mg, is generally administered. Daily dose for administration in humans for preventing this disease (cognitive disorder) could be in the range of about 0.01-10 mg/kg.

While the term for administering of at least one compound to prevent this disease (cognitive disorder) varies depending on species, and the nature and severity of the condition to be prevented, the compound may usually be administered to humans for a short term or a long term, i.e. for 1 day to 10 years.

Pharmaceutical compositions can be used in the preparation of individual, single unit dosage forms. The compounds of the present disclosure can be used in the form of pharmaceuticals compositions, for example, in solid, semisolid or liquid form, which contains one or more of the compounds according to the present disclosure as active ingredient associated with pharmaceutically acceptable carriers or excipient suitable for oral, parenteral such as intravenous, intramuscular, intrathecal, subcutaneous, enteral, intrarectal or intranasal administration. The active ingredient may be compounded, for example, with the usual non-toxic, pharmaceutically acceptable carriers for tablets, pellets, capsules, suppositories, solutions (saline for example), emulsion, suspensions (olive oil, for example), ointment and any other form suitable for use. The carriers which can be used are water, glucose, lactose gum acacia, gelatine, manitol, starch paste, magnesium trisilicate, corn starch, keratin, colloidal silica, potato starch, urea and other carriers suitable for use in manufacturing preparations, in solid, semisolid or liquid form, and in addition auxiliary, stabilizing, thickening and colouring agents and perfumes may be used. The active object compound is included in the pharmaceutical composition in an effective amount sufficient to prevent and/or treat the disease.

Single unit dosage forms of the disclosure are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of dosage forms of the disclosure will typically vary depending on their use. For example, a dosage form used in the acute treatment of a disease may contain larger amounts of one or more of the active agents it comprises than a dosage form used in the chronic treatment of the same disease. Similarly, a parenteral dosage form may contain smaller amounts of one or more of the active agents it comprises than an oral dosage form used to treat the same disease. These and other ways in which specific dosage forms encompassed by this disclosure will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active agents in the dosage form. For example, the decomposition of some active agents may be accelerated by some excipients such as lactose, or when exposed to water. Active agents that comprise primary or secondary amines are particularly susceptible to such accelerated decomposition. Consequently, this disclosure encompasses pharmaceutical compositions and dosage forms that contain little, if any, lactose or other mono- or di-saccharides. As used herein, the term “lactose-free” means that the amount of lactose present, if any, is insufficient to substantially increase the degradation rate of an active ingredient.

Lactose-free compositions of the disclosure can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This disclosure further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprise a primary or secondary amine are preferably anhydrous if substantial contact with moisture and/or humidity during manufacturing, packaging, and/or storage is expected. An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g. vials), blister packs, and strip packs.

The disclosure further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active agents in a dosage form may differ depending on factors such as, but not limited to, the route by which it is to be administered to patients. However, typical dosage forms of the disclosure comprise a compound according to the present disclosure or a pharmaceutically acceptable salt, solvate, hydrate, stereoisomer, clathrate, or prodrug thereof in an amount of from about 0.10 to about 150 mg. Typical dosage forms comprise a compound according to the present disclosure or a pharmaceutically acceptable salt, solvate, hydrate, stereoisomer, clathrate, or prodrug thereof in an amount of about 0.1, 1, 2, 5, 7.5, 10, 12.5, 15, 17.5, 20, 25, 50, 100 or 150 mg. In a particular embodiment, a preferred dosage form comprises a compound according to the present description in an amount of about 2, 5, 10, 25 or 50 mg. In a specific embodiment, a preferred dosage form comprises a compound according to the present description in an amount of about 5, 10, 25 or 50 mg.

Oral Dosage Forms of pharmaceutical compositions of the disclosure that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the disclosure are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms {e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the disclosure include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives {e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, {e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM. Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the disclosure is typically present in from about 50 to about 99 weight percent of the pharmaceutical composition or dosage form.

Disintegrants are used in the compositions of the disclosure to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the disclosure. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Piano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

A preferred solid oral dosage form of the disclosure comprises a compound of the disclosure, anhydrous lactose, microcrystalline cellulose, polyvinylpyrrolidone, stearic acid, colloidal anhydrous silica, and gelatin.

Active ingredients of the disclosure can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and U.S. Pat. Nos. 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein can be readily selected for use with the active ingredients of the disclosure. The disclosure thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

Parenteral dosage forms can be administered to patients by various routes including, but not limited to, subcutaneous, intravenous (including bolus injection), intramuscular, and intraarterial. Because their administration typically bypasses patients' natural defences against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. Suitable vehicles that can be used to provide parenteral dosage forms of the disclosure are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the disclosure. For example, cyclodextrin and its derivatives can be used to increase the solubility of a compound of the disclosure and its derivatives. See, e.g., U.S. Pat. No. 5,134,127, which is incorporated herein by reference.

Topical and mucosal dosage forms of the disclosure include, but are not limited to, sprays, aerosols, solutions, emulsions, suspensions, eye drops or other ophthalmic preparations, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients {e.g., carriers and diluents) and other materials that can be used to provide topical and mucosal dosage forms encompassed by this disclosure are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

Typically, active ingredients of the disclosure are preferably not administered to a patient at the same time or by the same route of administration. This disclosure therefore encompasses kits which, when used by the medical practitioner, can simplify the administration of appropriate amounts of active ingredients to a patient.

A typical kit of the disclosure comprises a dosage form of a compound of the disclosure, or a pharmaceutically acceptable salt, solvate, hydrate, stereoisomer, prodrug, or clathrate thereof. Kits encompassed by this disclosure can further comprise additional active agents. Examples of the additional active agents include, but are not limited to, those disclosed herein. Kits of the disclosure can further comprise devices that are used to administer the active ingredients. Examples of such devices include, but are not limited to, syringes, drip bags, patches, and inhalers.

Kits of the disclosure can further comprise cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

The following examples and methods are offered for illustrative purposes only, and are not intended to limit the scope of the present disclosure in any way.

### METHODS AND EXAMPLES

A series of non-clinical pharmacology and toxicology studies have been performed to support the clinical evaluation of the compounds according to the present disclosure in human subjects. These studies were performed in accordance with internationally recognized guidelines for study design and in compliance with the requirements of Good Laboratory Practice (GLP) unless otherwise noted.

### Example 1: The Effects of AP-61 in Wistar Rats in a Test of Natural Forgetting in the Object Recognition Task (ORT)

In example 1, the compound AP-61 a 7-(4-tert butyl-cyclohexyl)-imidazotriazione (a compound of the formula II) was tested in the ORT in 3-4 month old male Wistar rats using a 24-h interval between the trials to induce natural forgetting.

One group of twenty four 3-4 month old male Wistar rats (Charles River, Sulzfeld, Germany) were used (average body weight at the beginning of the study: 365 g). All animals were housed individually in standard green line Tecniplast IVC cages on sawdust bedding. The animals were housed on a reversed 12/12-h light/dark cycle (lights on from 19:00 h to 07:00 h) and had free access to food and water. The rats were housed and tested in the same room. A radio, playing softly, provided background noise in the room. All testing was performed between 09:00 h and 18:00 h.

AP-61 was dissolved in 0.5% methylcellulose (MC) and 2% Tween 80. The solutions were prepared daily. Doses of 0.01, 0.03 and 0.1 mg/kg of AP-61 or vehicle were administered p.o. (injection volume was 2 ml/kg), immediately after T1. Of note, PDE4 inhibition effects only late consolidation in the ORT, i.e. at about 3 h after T1 (Rutten et al., 2007). Considering the long plasma Tmax of AP-61 (5 h), it was chosen to administer immediately after T1 to obtain highest plasma concentrations during the late consolidation phase.

The ORT was performed as described elsewhere (Ennacour and Delacour, 1988). The apparatus consisted of a circular arena, 83 cm in diameter. Half of the 40 cm high wall was made of gray polyvinyl chloride, the other half of transparent polyvinyl chloride. Fluorescent red tubes and a light bulb provided a constant illumination of about 20 lux on the floor of the apparatus.

Two objects were placed in a symmetrical position about 10 cm away from the gray wall. Four objects were used: 1) a standard 1 L brown transparent glass bottle (diameter 10 cm, height 22 cm) filled with water, 2) a metal cube (10.0×5.0×7.5 cm) with two holes (diameter 1.9 cm), 3) a cone consisting of a gray polyvinyl chloride base (maximal diameter 18 cm) with a collar on top made of brass (total height 16 cm), and 4) an aluminum cube with a tapering top (13.0×8.0×8.0 cm). A rat could not displace the objects.

A testing session comprised two trials, each with durations of 3 min. During T1 the apparatus contained two identical objects (samples). A rat was always placed in the apparatus facing the wall at the middle of the front (transparent) segment. After T1 the rat was put back in its home cage for a 24-h interval. Subsequently, the rat was put back in the apparatus for T2, but now with a familiar object from T1 (the sample) and a new object. The times spent in exploring each object during T1 and T2 were recorded manually with a personal computer.

Exploration was defined as follows: directing the nose to the object at a distance of no more than 2 cm and/or touching the object with the nose. Sitting on the object was not considered as exploratory behavior. In order to avoid the presence of olfactory cues, the objects were thoroughly cleaned after each trial and three sets of objects were used. All combinations and locations of objects were used in a balanced manner to reduce possible biases due to preferences for particular locations of objects.

Prior to compound testing studies, the animals were handled daily, adapted to the procedure, and allowed to explore the apparatus. The rats were adapted to injections of saline and tested until they showed stably good discrimination performance at a 1-h interval and no discrimination at a 24-h interval.

The measures were the times spent by rats in exploring each object during T1 and T2. The time spent in exploring the two identical samples in T1 were represented by ‘a1’ and ‘a2’, respectively. The time spent in exploring the sample and the new object in T2 were represented by ‘a’ and ‘b’, respectively. From these exploration times the following variables were calculated: e1, e2 and d2 (Table 1). The minimum level of exploration needed for a reliable memory performance is 7 s (Akkerman et al., 2012). When exploration was below this cut-off, rats were removed from the analysis. Furthermore, the d2 index is a relative measure of discrimination corrected for exploratory activity. The d2 index can range from −1 to 1, with −1 or 1 indicating complete preference for the familiar or novel object, respectively, and 0 signifying no preference for either object.

TABLE 1
Derived Measures in the Object Recognition Task

| Trial number | Exploration time (s) | Discrimination index |
|---|---|---|
| T1 | e1 = a1 + a2 | Not determined |
| T2 | e2 = a + b | d2 = (b − a)/e2 |

One-sample t-statistics could be performed in order to assess whether the d2 index for each treatment group differed significantly from zero/chance level. However, comparison of the value of d2 with the value zero with no variance is not the most suitable way of analyzing object recognition since there is an increased chance of making a type I error. Therefore, comparing the treatment groups with a fictive group showing no discrimination is a widely used method for statistical analysis of the ORT. The fictive group was constructed based on previous reports and has a d2 of 0 and SEM of 0.07 (Akkerman et al., 2012b). Treatment groups, excluding the fictive group, were also compared using one-way ANOVAs. When the overall ANOVA was significant, a post-hoc analysis with Bonferroni t-tests (all pairwise comparisons) was performed. An a level of 0.05 was considered significant.

The results of exploration times in T1 and T2 and the discrimination index of different doses of AP-61, administered p.o., immediately after learning, are summarized in Table 2. There were no differences between treatment conditions in the level of exploration in T1 (e1: F(3,81)=0.75, n.s.) and T2 (e2: F(3,81)=0.39, n.s.).

TABLE 2
Means (±SEM) for the derived measures in the ORT for the effect
of AP61 in male Wistar rats

| Dose of AP61 | | | | |
|---|---|---|---|---|
| (p.o., mg/kg) | e1 (s) | e2 (s) | d2 | n |
| Vehicle | 22.09 (2.6) | 21.77 (2.04) | 0.06 (0.08) | 22 |
| 0.01 | 21.88 (1.78) | 24.54 (1.79) | 0.10 (0.09) | 20 |
| 0.03 | 18.88 (1.48) | 22.58 (1.69) | 0.25 (0.07)* | 22 |
| 0.1 | 22.44 (1.55) | 22.34 (2.01) | 0.01 (0.08) | 21 |

The delay interval between T1 and T2 was 24 h. e1, total exploration time during T1; e2, total exploration time during T2; d2, discrimination index between the new and familiar objects for T2; n, group size. A t-test showed that d2 index differed from the fictive group showing no discrimination (d2=0, SEM=0.07), * p<0.05.

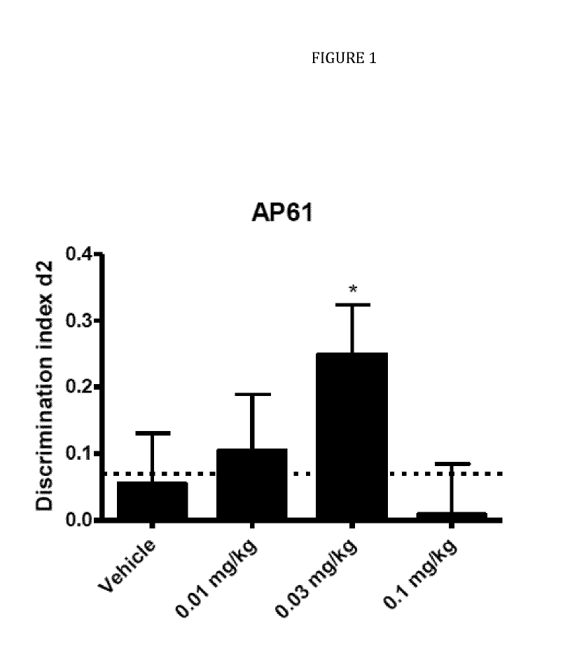

A t-test comparing the d2 (zero) of the fictive group with the d2 of the treatment groups showed that 0.03 mg/kg differed significantly from the fictive group (t(44)=2.57, p<0.05) (FIG. 1). Since 0.03 mg/kg AP61 differed significantly from the fictive group, it can be concluded that this dose had an effect for enhancing memory in the ORT.

### Example 2: The Effects of AP-61 and Rolipram in the Xylazine/Ketamine-Induced Anesthesia Test in Male Wistar Rats

Development of PDE4-Is as therapeutic drugs has always been hampered by the dose-limiting emetic side effects (nausea and vomiting) in humans of the classic PDE4-I rolipram, which has been developed as a possible anti-depressant in the eighties of the previous century (Prickaerts, 2010). Currently, PDE4-Is are being developed which show a strong reduction in emetic side effects. In the present study, the possible emetic properties of AP-61, were investigated and compared with the emetic properties of the classic PDE4-I rolipram.

The mechanism of the emetic response associated with PDE4-Is is thought to be a consequence of the inhibition of PDE4 in non-target tissues. It is believed that PDE4-Is produce a pharmacological response analogous to that of presynaptic α2-adrenoreceptor inhibition by elevating intracellular levels of cAMP in noradrenergic neurons. Therefore, by removing an inhibitory mechanism, PDE4-Is are thought to modulate the release of mediators including 5-HT, substance P and noradrenaline involved in the onset of the emetic reflex mediated at emetic brainstem centers. PDE4-Is have the ability to reverse α2-adrenoreceptor agonist-mediated anesthesia with xylazine/ketamine in rodents (Robichaud et al., 2002). This effect is very likely mediated at the locus coeruleus in the brain stem. This confirms the postulate that PDE4-Is have effects similar to those of a2-adrenoreceptor antagonists. Since rodents are non-vomiting species, the ability of a PDE4-I to shorten a2-adrenergic receptor-mediated xylazine/ketamine anesthesia is therefore used as well-established surrogate measure of emesis in rodents.

Twenty four 3-4 month old male Wistar rats (Charles River, Sulzfeld, Germany) were used (average body weight at the beginning of the study: 365 g). All animals were housed individually in standard green line Tecniplast IVC cages on sawdust bedding. The animals were housed on a reversed 12/12-h light/dark cycle (lights on from 19:00 h to 07:00 h) and had free access to food and water. The rats were housed and tested in the same room. A radio, playing softly, provided background noise in the room. All testing was performed between 09:00 h and 18:00 h.

AP-61 was dissolved in 0.5% methylcellulose (MC) and 2% Tween 80. Doses of 0.03, 0.1, 0.3 and 1.0 mg/kg of AP-61 or vehicle were administered p.o. (injection volume was 2 ml/kg). The highest dose of 3.0 mg/kg AP-61 was dissolved in 0.5% MC and 6% tween 80 to improve solubility at this high concentration. The emetic properties of the PDE4-I rolipram are already assessed (Bruno et al., 2011). In the current study rolipram was used as a reference compound for AP-61 and applied in at dosages of 0.1 and 0.3 mg/kg. Rolipram was dissolved in 0.5% MC and 2% tween 80 (injection volume 2 ml/kg, route of administration was p.o.). For the induction of anesthesia, 10 mg/kg ketamine (Eurovet Animal Health, The Netherlands) and 10 mg/kg xylazine (CEVA Santé Animale, The Netherlands) were used (both administered i.p.). All solutions were prepared daily.

Rats were anesthetized with a combination of xylazine and ketamine (both 10 mg/kg, i.p). Fifteen minutes after induction of the anesthesia, rats were treated with rolipram or vehicle (0.1 or 0.3 mg/kg, p.o.) and the animals were placed in a dorsal position. The restoration of the righting reflex, i.e. when the animal no longer remained on its back and turned itself spontaneously to the prone position, was used as an endpoint to determine the duration of anesthesia. Animals that were not anesthetized after 15 min were excluded from the analysis.

AP-61 reached peak concentration at 5 h after oral administration. Because of this, induction of anesthesia with the combination of xylazine and ketamine was done 3.5 h after oral administration of AP-61. Animals were then placed in a dorsal position. The time delay to the recovery of the righting reflex was used as an endpoint to measure the duration of the anesthesia. Again, animals that were not anesthetized after 15 min were excluded from the analysis.

The restoration of the righting reflex, i.e. when the animal no longer remained on its back and turned itself spontaneously to the prone position (standing on four paws), was used as an endpoint to determine the duration to the anesthesia. Each test day a vehicle group was included. Outliers were removed from the analysis (Dixon-test). Each daily vehicle was set at 100% while the other treatment conditions of that day were expressed as a percentage of the vehicle. The following formula was used:

Duration of anesthesia after drug treatment (min)×(100/mean of duration of anesthesia of vehicle treatment (min))

Statistical significance between treatment conditions was calculated using a one-way ANOVA followed by Bonferroni post-hoc comparison test. An α level of 0.05 was considered significant.

The effect of 0.1 mg/kg and 0.3 mg/kg rolipram (p.o.) on the recovery times after xylazine/ketamine-induced anesthesia is shown in 3. Vehicle treatment was set at 100%.

TABLE 3
Means (±SEM) of the relative recovery times of rolipram in the
xylazine/ketamine-induced anesthesia in male Wistar rats.

| Dose level of | | | |
|---|---|---|---|
| rolipram (mg/kg) | Mean | SEM | n |
| Vehicle | 100.00 | 5.79 | 6 |
| 0.1 | 84.58 | 3.45 | 7 |
| 0.3 | 69.34 | 1.59 | 6 |

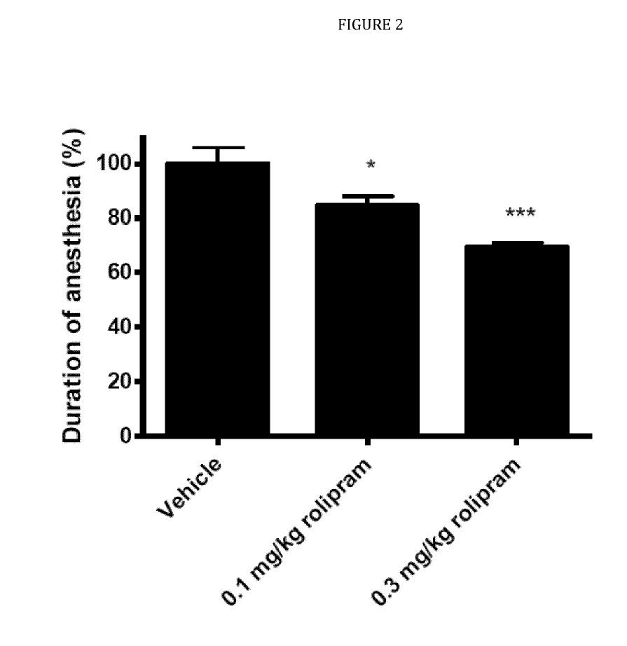

Rolipram treatment significantly affected the duration of xylazine/ketamine-induced anesthesia (F(2,16)=14.27, p<0.001). Post-hoc analysis indicated that the duration of the xylazine/ketamine-induced anesthesia of animals treated with 0.1 and 0.3 mg/kg rolipram was significantly reduced when compared to the vehicle treated animals (p<0.05 for 0.1 mg/kg rolipram, p<0.001 mg/kg for 0.3 mg/kg rolipram; FIG. 2). In addition, both doses of rolipram also differed significantly from each other (p<0.05).

The effect of different doses of AP61 (p.o.) on the recovery times after xylazine/ketamine-induced anesthesia is shown in 4. Vehicle treatment was set at 100%.

TABLE 4
Means (±SEM) of the relative recovery times of AP61 in the
xylazine/ketamine-induced anesthesia in male Wistar rats.

| Dose level of AP61 | | | |
|---|---|---|---|
| (mg/kg) | Mean | SEM | n |
| Vehicle | 100.00 | 3.87 | 21 |
| 0.03 | 97.16 | 8.34 | 6 |
| 0.1 | 107.16 | 16.42 | 7 |
| 0.3 | 154.09 | 20.44 | 6 |
| 1.0 | 193.29 | 27.19 | 5 |
| 3.0 | 108.37 | 10.99 | 10 |

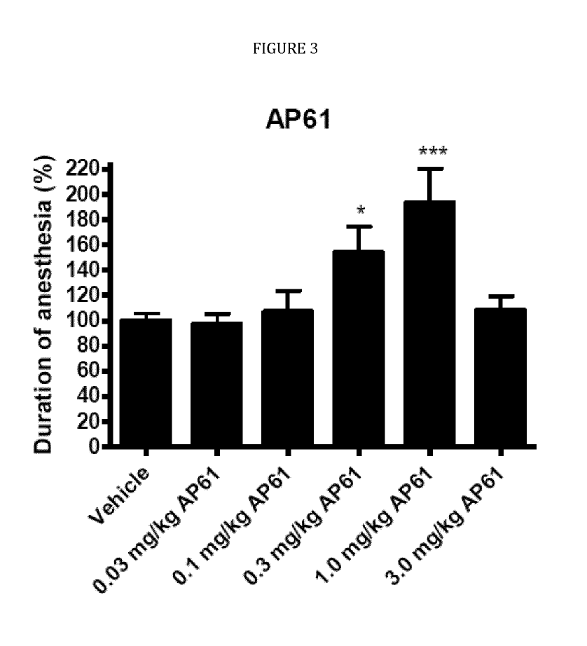

When comparing between groups by use of a one-way ANOVA, significant differences between the duration of xylazine/ketamine-induced anesthesia of different doses of AP61 were found (F(5,49)=7.99, p<0.001; FIG. 3). Post-hoc analysis indicated that the duration of anesthesia of animals treated with 0.3 mg/kg and 1.0 mg/kg AP61 was prolonged compared to vehicle treated animals (p<0.05 for 0.3 mg/kg and p<0.001 for 1.0 mg/kg). There were also significant differences of the duration of anesthesia between the 1.0 mg/kg AP61 and 0.03, 0.1 and 3.0 mg/kg AP61 (p<0.01).

The study results show that the oral administration of rolipram (2 mg/kg, 15 min after induction of anesthesia) led to a significant reduction of the duration of the xylazine/ketamine-induced anesthesia in male Wistar rats (FIG. 2). For AP-61, none of the doses led to a shortened duration of xylazine/ketamine-induced anesthesia compared with vehicle (FIG. 3). From this it can be concluded that none of the different doses of AP-61 showed emetic properties Thus, AP-61 is a carefully selected PDE4-I which does not show any signs of gastrointestinal toxicity within the effective dose range by which AP-61 clearly differentiates from the classical PDE4-I like rolipram.

### Example 3: The Effect of Co-Administration of Sub-Efficacious Doses of AP61 (0.01 mg/kg) and Donepezil (0.1 mg/kg) in Wistar Rats in a Test of Natural Forgetting in the ORT

In the third example a combination of sub-efficacious doses of donepezil (0.1 mg/kg) and AP-61 (0.01 mg/kg) were investigated. AP61 was dissolved in 0.5% methylcellulose (MC) and 2% Tween 80. The solutions were prepared daily. Doses of 0.01, 0.03 and 0.1 mg/kg of AP61 or vehicle were administered p.o. (injection volume was 2 ml/kg), 4 min after T1. Considering the long plasma Tmax of AP61 (5 h), it was chosen to administer 4 min after T1 to obtain highest plasma concentrations during the late consolidation phase. Donepezil was dissolved in saline and also prepared daily. A dose of 0.1 mg/kg was administered p.o. (injection volume 2 ml/kg), 30 min before T1 to mainly target the memory acquisition process in the ORT.

The results of exploration times in T1 and T2 and the discrimination indexes of the different conditions are summarized in Table 5. 0.01 mg/kg AP61 or vehicle was combined with 0.1 mg/kg donepezil or its vehicle, administration was 4 min after and 30 min before T1, respectively. There were no differences between treatment conditions in the level of exploration in T1 (e1: F(3,69)=1.10, n.s.) and T2 (e2: F(3,69)=1.12, n.s.).

TABLE 5
Means (±SEM) for the derived measures in the object recognition task
for the effect of sub-efficacious doses of AP61 and donepezil in
male Wistar rats.

| Treatment | | | | |
|---|---|---|---|---|
| condition | e1 (s) | e2 (s) | d2 | n |
| Vehicle + vehicle | 49.49 (1.93) | 45.46 (2.32) | −0.03 (0.06) | 24 |
| AP61 + vehicle | 45.00 (1.77) | 47.45 (2.49) | 0.02 (0.05) | 24 |
| Vehicle + | 45.72 (1.89) | 46.54 (1.62) | 0.04 (0.04) | 24 |
| donepezil | | | | |
| AP61 + donepezil | 47.15 (2.05) | 49.77 (2.06) | 0.31 (0.04)### | 24 |

AP61 administration was 4 min after T1. Donepezil was administered 30 min before T1. The delay interval between T1 and T2 was 24 h. e1, total exploration time during T1; e2, total exploration time during T2; d2, discrimination index between the new and familiar objects for T2; n, group size. A t-test showed that d2 index differed from the fictive group showing no discrimination (d2=0, SEM=0.07), ### p<0.001.

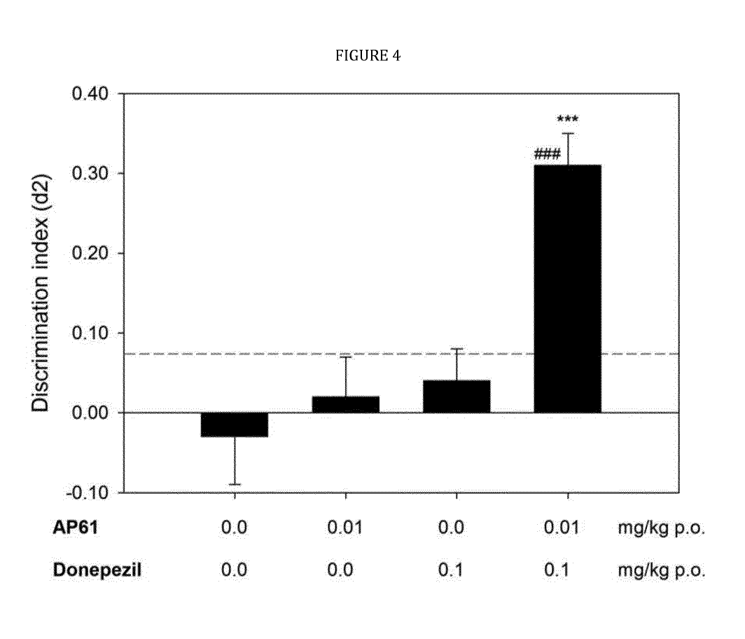

A t-test comparing the d2 (zero) of the fictive group with the d2 of the treatment groups showed that the combination of 0.01 mg/kg AP61 and 0.1 mg/kg donepezil differed significantly from the fictive group (t(46)=4.04, P=0.000, FIG. 4). In addition, the repeated-measures ANOVA revealed an effect for the discrimination index (d2) (F(3,69)=8.43, P=0.000, FIG. 4). Post hoc t-tests indicated a significantly higher discrimination performance in the 0.01 mg/kg AP61 and 0.1 mg/kg donepezil combined condition when compared to the vehicle condition. From this it can be concluded that the combination of sub-efficacious doses of AP61 and donepezil, which had no effect when administered separately, fully restored memory performance of rats in the ORT.

1-14. (canceled)

15. A method for treating diminished cognitive processes in cognitive, concentration capacity, learning capacity and/or memory retentiveness disorders in a patient, which comprises administering a therapeutically effective amount of a pharmaceutical composition, wherein the composition comprises a compound of the formula I: 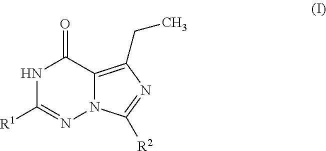
wherein:
i) R¹ denotes (C₆-C₁₀)-aryl, which is optionally substituted by identical or different residues selected from the group consisting of halogen, (C₁-C₄)-alkyl, tri fluoromethyl, cyano, nitro und trifluoromethoxy, or denotes (C₁-C₈)-alkyl, which is optionally substituted by 3- to 10-membered carbocyclyl, or denotes 3- to 10-membered carbocyclyl, which is optionally substituted by identical or different (C₁-C₄)-alkyl residues, and R² denotes 4-tert-butyl-cyclohex-1-yl, or
ii) R¹ denotes naphthyl, or denotes phenyl, which is optionally substituted by identical or different halogen atoms, and R² denotes 4-tert-butyl-cyclohex-1-yl, or
iii) R¹ denotes (C₆-C₁₀)-aryl, which is optionally substituted by identical or different residues selected from the group consisting of halogen, (C₁-C₄)-alkyl, trifluoromethyl, cyano, nitro und trifluoromethoxy, or denotes (C₁-C₈)-alkyl, which is optionally substituted by 3- to 10-membered carbocyclyl, or denotes 3- to 10-membered carbocyclyl, which is optionally substituted by identical or different (C₁-C₄)-alkyl residues, and R² denotes cis-4-tert-butylcyclohex-1-yl, or
iv) R¹ denotes naphthyl, or denotes phenyl, which is optionally substituted by identical or different halogen atoms, and R² denotes cis-4-tert-butylcyclohex-1-yl.

16. The method according to claim 15, wherein the compound is a 7-(4-tert-butylcyclohexyl)-imidazotriazinone.

17. The method according to claim 15, wherein the compound is a compound of the formula (II) 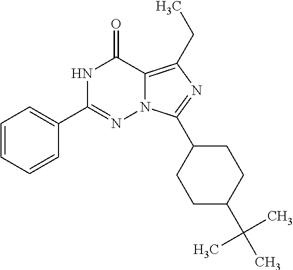 [SMILES: CCc1nc(C2CCC(C(C)(C)C)CC2)n2nc(-c3ccccc3)[nH]c(=O)c12]
or a pharmaceutically acceptable salt, solvate or stereoisomer thereof.

18. The method according to claim 17, wherein the stereoisomer of the compound is the R or S enantiomer.

19. The method according to claim 15, wherein said compound is administered at daily dosages between 0.1 mg-150 mg/body, preferable 1 mg-100 mg/body and more preferable 2 mg-50 mg/body.

20. The method according to claim 15, wherein the diminished cognitive processes is experienced in several patient groups, e.g. by schizophrenic, depressive or psychotic patients and patients with attention deficit hyperactivity disorder (ADHD), Parkinson's disease, mild cognitive impairment (MCI), dementia, anxiety, age associated memory impairment, Alzheimer's Disease or post-traumatic stress disorder and in a range of neurodegenerative diseases in addition to Parkinson's Disease and Alzheimer's Disease.

21. The method according to claim 15, wherein the diminished cognitive processes refer to the difficulties with attention, learning, memory and executive function (relevant reactions to external stimuli). These can include: deficits in attention, disorganized thinking, slow thinking, difficulty in understanding, poor concentration, impairment of problem solving, poor memory, difficulty in expressing thoughts and/or difficulty in integrating thoughts, feelings and behaviour and extinction of irrelevant thoughts as well as attention and vigilance, verbal learning and memory, visual learning and memory, speed of processing and social cognition.

22. The method according to claim 15, wherein the patient is a human.
